(19) United States
(12) Patent Application Publication
(10) Pub. No.: US 2005/0085409 A1
(43) Pub. Date: Apr. 21, 2005

(54) Particle formation

(76) Inventors: Andreas Kordikowski, Near Skipton (GB); Darren John Gilbert, Bradford (GB)

Correspondence Address: NEKTAR THERAPEUTICS, 150 INDUSTRIAL ROAD, SAN CARLOS, CA, 94070, US

(21) Appl. No.: 10/753,757

(22) Filed: Jan. 7, 2004

(30) Foreign Application Priority Data
Jan. 8, 2003 (GB) 0200339.9

(51) Int. Cl. A61K 38/00; B29B 9/00

(52) U.S. Cl. 514/2; 264/5

(57) ABSTRACT

Method for preparing a target substance in particulate form, by introducing into a particle formation vessel, through separate first and second fluid inlets respectively, (a) a solution or suspension of the target substance in a fluid vehicle (the “target solution/suspension”) and (b) a compressed fluid anti-solvent for the substance, and allowing the anti-solvent to extract the vehicle from the target solution/suspension so as to form particles of the target substance, wherein the target solution/suspension enters the vessel downstream of the point of entry of the anti-solvent and at a point which lies on or close to the main axis of anti-solvent flow, and wherein the anti-solvent has a sub-sonic velocity as it enters the particle formation vessel.

### FIELD OF THE INVENTION

This invention relates to methods for use in forming particles of a target substance, and to their particulate products.

### BACKGROUND OF THE INVENTION

It is known to use a compressed fluid, typically a supercritical or near-critical fluid, as an anti-solvent to precipitate particles of a substance of interest (a “target substance”) from solution or suspension. The basic technique is known as “GAS” (Gas Anti-Solvent) precipitation [Gallagher et al, “Supercritical Fluid Science and Technology”, ACS Symp. Ser., 406, p334 (1989)]. Versions of it have been disclosed for instance in EP-0 322 687 and WO-90/03782.

In one particular version known as the Nektar™ SCF particle formation process (previously known as SEDS™ or “Solution Enhanced Dispersion by Supercritical fluids”), a target substance is dissolved or suspended in an appropriate fluid vehicle, and the resulting “target solution/suspension” then co-introduced into a particle formation vessel with an anti-solvent fluid (usually supercritical) in which the vehicle is soluble. The co-introduction is effected in a particular way, such that:
the target solution/suspension and the anti-solvent both meet and enter the vessel at substantially the same point; and
at that point, the mechanical energy of the anti-solvent serves to disperse the target solution/suspension (ie, to break it up into individual fluid elements) at the same time as the anti-solvent extracts the vehicle so as to cause particle formation.

Thus, in the Nektar™ SCF process, the compressed fluid serves not only as an anti-solvent but also as a mechanical dispersing agent. The simultaneity of fluid contact, dispersion and particle formation provides a high degree of control over the physicochemical properties of the particulate product.

Versions of this process are described in WO-95/01221, WO-96/00610, WO-98/36825, WO-99/44733,WO-99/59710,WO-01/03821,WO-01/15664 and WO-02/38127. Other “SEDS™”-based processes are described in WO-99/52507, WO-99/52550, WO-00/30612, WO-00/30613, WO-00/67892 and WO-02/58674.

Another version of the GAS technique is described in WO-97/31691, in which a special form of two-fluid nozzle is used to introduce a “target solution/suspension” and an energising gas into a particle formation vessel containing a supercritical anti-solvent. The energising gas can be the same as the anti-solvent fluid. Within the nozzle, a restriction generates sonic waves in the energising gas/anti-solvent flow and focuses them back (ie, in a direction opposite to that of the energising gas flow) on the outlet of the target solution/suspension passage, resulting in mixing of the fluids within the nozzle before they enter the particle formation vessel. It is suggested that where the energising gas is the same as the anti-solvent (typically supercritical carbon dioxide), its flow rate could be sufficiently high to obtain a sonic velocity at the nozzle outlet. However, the authors do not appear ever to have achieved such high velocities in their experimental examples.

Other modifications have been made to the basic GAS process in order to affect atomisation of the target solution/suspension at the point of its contact with the compressed fluid anti-solvent. For example, U.S. Pat. No. 5,770,559 describes a GAS precipitation process in which a target solution is introduced, using a sonicated spray nozzle, into a pressure vessel containing a supercritical or near-critical anti-solvent fluid-see also Randolph et al in Biotechnol. Prog., 1993, 9, 429-435.

Our co-pending PCT patent application published as WO-03/008082 describes a version of the Nektar™ SCF GAS process in which a target solution/suspension and a compressed fluid anti-solvent are introduced separately into a particle formation vessel through first and second fluid inlets respectively. The outlet of the first fluid inlet is positioned downstream of, and directly in line with, that of the second inlet, so that the target solution/suspension is introduced directly into the anti-solvent flow. The anti-solvent has a near-sonic, sonic or supersonic velocity as it enters the vessel, necessitating the use of high back pressures across the second fluid inlet and also the pre-heating of the anti-solvent to compensate for its Joule-Thomson cooling as it expands into the vessel.

It has now surprisingly been found that such a particle formation process may also be practised to good effect using lower (ie, sub-sonic) anti-solvent velocities. Although the anti-solvent and target solution/suspension are not co-introduced through the same fluid inlet, nevertheless the fluid inlet arrangement of WO-03/008082 (previously only described for use in conjunction with sonic anti-solvent velocities) appears to allow a high degree of control over the product properties, enabling the formation of fine particles with narrow size distributions in a similar manner to the basic Nektar™ SCF process.

The present invention thus provides alternative GAS-based particle formation techniques which can yield advantages comparable to if not better than those of the prior art processes and overcome some of the problems associated with certain prior art techniques.

### STATEMENTS OF THE INVENTION

According to a first aspect of the present invention there is provided a method for preparing a target substance in particulate form, the method comprising introducing into a particle formation vessel, through separate first and second fluid inlets respectively, (a) a solution or suspension of the target substance in a fluid vehicle (the “target solution/suspension”) and (b) a compressed fluid anti-solvent for the substance, and allowing the anti-solvent fluid to extract the vehicle from the target solution/suspension so as to form particles of the target substance, wherein the target solution/suspension enters the vessel downstream of the point of entry of the anti-solvent fluid and at a point which lies on or close to the main axis of anti-solvent flow out of the second fluid inlet, and wherein the anti-solvent fluid has a sub-sonic velocity as it enters the particle formation vessel.

The target solution/suspension and the anti-solvent must therefore be introduced separately into the particle formation vessel and contact each other downstream of (preferably immediately downstream of) the point of anti-solvent entry into the vessel.

By “sub-sonic velocity” is meant that the velocity of the anti-solvent fluid as it enters the vessel is lower than the velocity of sound in that fluid at that point. “Sub-sonic velocity” does not encompass a “near-sonic” velocity which is slightly lower than, but close to, the velocity of sound in that fluid at that point. Thus for instance for the fluid to be travelling with a sub-sonic velocity, its “Mach number” M (the ratio of its actual speed to the speed of sound) will be 0.8 or lower, preferably 0.7 or lower. Generally speaking, in the method of the invention, the Mach number for the anti-solvent fluid on entering the particle formation vessel may be from 0.05 to 0.5, more preferably from 0.1 to 0.4.

(References in this specification to a fluid entering a vessel are to the fluid exiting an inlet means (for example, a nozzle) used to introduce the fluid into the vessel. For these purposes, therefore, the inlet means is to be considered as upstream of the vessel in the direction of fluid flow, although parts of it (in particular its outlet) may be located physically within the vessel.)

The method of the invention has been found in many cases to provide a high degree of control over physicochemical product characteristics, in particular size and size distribution, but without the need for the near-sonic, sonic or supersonic velocities required in the method of WO-03/008082. This is surprising in the context of the GAS art as a whole, since generally such high levels of control have been achievable only by co-introducing the anti-solvent and target solution/suspension through a common inlet, for instance the two-component coaxial nozzle described in the examples of WO-95/01221, or alternatively by separating the two fluid inlets but compensating by using the extremely high anti-solvent velocities taught by WO-03/008082.

If high anti-solvent velocities are to be used in the method of the invention, there may need to be a drop in pressure as the anti-solvent enters the particle formation vessel. This can typically be achieved by imparting a relatively high “back pressure” to the anti-solvent, for example by using a high anti-solvent flow rate and forcing it through a restriction such as a nozzle into a vessel maintained at a significantly lower pressure.

However, such pressure reduction can cause undesirable Joule-Thomson cooling of the anti-solvent. Accordingly, the temperature of the anti-solvent upstream of the particle formation vessel needs to be sufficiently high that the fluid remains at an appropriate temperature (typically above its critical temperature T_{C}), even after expanding into the vessel. The method of the invention thus may include pre-heating the anti-solvent fluid, upstream of the particle formation vessel, to a temperature sufficient to compensate for its Joule-Thomson cooling as it enters the vessel.

Thus, in the method of the invention it may be desirable that (i) the pressure in the particle formation vessel is P₁ which is preferably greater than the critical pressure P_{c} of the anti-solvent, (ii) the anti-solvent is introduced through a restricted inlet so as to have a back pressure of P₂, where P₂ is typically greater than P₁, (iii) the temperature in the particle formation vessel is T₁ which is preferably greater than the critical temperature T_{c} of the anti-solvent, (iv) the anti-solvent is introduced into the vessel at a temperature T₂, where T₂ is greater than T₁, (v) T₁ and T₂ are such that Joule-Thomson cooling of the anti-solvent as it enters the vessel does not reduce the anti-solvent temperature to below that required of it at the point of particle formation (and are preferably such that the anti-solvent temperature does not fall below T_{c} within the vessel) and (vi) P₁, P₂, T₁ and T₂ are such that the anti-solvent fluid has a sub-sonic velocity as it enters the particle formation vessel.

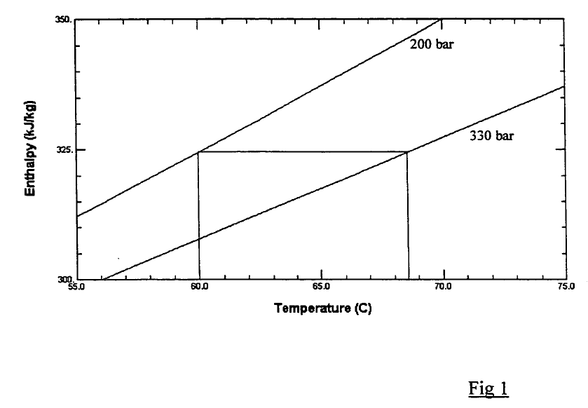

Any anti-solvent expansion on entering the particle formation vessel will be isenthalpic. Thus, an appropriate temperature for the anti-solvent upstream of the vessel may be derived from enthalpy charts for the fluid, for instance as illustrated for carbon dioxide in FIG. 1. (For CO₂, the critical temperature T_{c} is 31° C. (304 K) and the critical pressure P_{c} is 74 bar.) FIG. 1 shows how, when working with a pressure reduction from 330 to 200 bar for the CO₂ on entering a particle formation vessel, the upstream temperature should be at least about 341.5 K (68.5° C.) to achieve an appropriate temperature of say 333 K (60° C.) or greater when the CO₂ enters the vessel.

The pressures and temperatures needed to ensure a sub-sonic velocity depend on the nature of the anti-solvent fluid. In the case of a carbon dioxide anti-solvent, for instance, in order to achieve a sub-sonic velocity the operating conditions must satisfy the formula:

$$\frac{{p}_{0}}{{p}_{1}} \geq {( \frac{2}{k + 1} )}^{k / ( k - 1 )}$$

where p1 is the total CO₂ pressure upstream of entry into the particle formation vessel (ie, the CO₂ back pressure plus the pressure in the vessel) and pₒ is the CO₂ pressure immediately on entry into the vessel, and k is the ratio of the specific heats of CO₂ at constant pressure (Cₚ) and constant volume (Cᵥ) [see, for instance, International Thermodynamic Tables of the Fluid State, Angus et al, Pergamon Press, 1976, or the standard reference data program of the National Institute of Standards and Technology (NIST), Gaithersburg, USA].

An alternative method for calculating the anti-solvent velocity is from its volumetric flow rate and the area of the outlet of the second fluid inlet (typically a nozzle) through which the anti-solvent is introduced.

The desired anti-solvent velocity is ideally achieved simply by the use of appropriate anti-solvent flow rates, back pressures and/or operating temperatures, and without the aid of mechanical, electrical and/or magnetic input such as for example from impellers, impinging surfaces especially within the anti-solvent inlet, electrical transducers and the like. Introducing the anti-solvent via a convergent nozzle, ideally as a single fluid stream, may also help in the achievement of appropriate fluid velocities. Further “energising” fluid streams, such as those required in the method of WO-97/31691, are not then needed in order to achieve the desired level of control over the contact between the target solution/suspension and the anti-solvent fluid.

The use of a fluid inlet arrangement as described above in connection with the method of the invention can allow achievement of smaller particle sizes and narrower size distributions than in many prior art GAS-based particle formation processes, even sometimes than in earlier Nektar™ SCF™-type processes. In particular it can allow the formation of small micro- or even nano-particles, for instance of volume mean diameter 9 μm or less, preferably 5 μm or less, more preferably 2 μm or less, yet more preferably 1 μm or 900 nm or 600 nm or 500 nm or less. Such particulate products preferably have narrow size distributions, such as with a particle size spread of 2.5 or less, preferably 2.2 or less, more preferably 2.1 or 2.0 or less. (Particle size “spread” is defined as (D₉₀-D₁₀)/D₅₀ where D is the volume mean diameter of the relevant particle population.)

Particle sizes may be measured for instance using (a) an Aerosizer™ time-of-flight instrument (which gives an aerodynamic equivalent particle diameter, MMAD) or (b) a laser diffraction sensor such as the Helos™ system available from Sympatec GmbH, Germany (which provides a geometric projection equivalent MMD). Volume mean diameters may be obtained in both cases using commercially available software packages.

The inlet arrangement used in the present invention also appears to lead to more efficient vehicle extraction, thus potentially yielding particles with lower residual solvent levels and generally lower levels of impurities. A particulate product prepared according to the invention will typically contain less than 2000 ppm of residual solvent. It preferably contains less than 1000 or 500 ppm, more preferably less than 200 ppm, most preferably less than 150 or 100 or even 50 ppm residual solvent, by which is meant solvent(s) which were present at the point of particle formation, for instance in the target solution/suspension and/or the anti-solvent fluid. Still more preferably the product contains no detectable residual solvent, or at least only levels below the relevant quantification limit(s).

Generally such a product will preferably contain 2.5% w/w or less, more preferably 2 or 1.5 or 1% w/w or less of impurities, by which is meant substances (either solid or liquid phase) other than the target substance(s) intended to be formed into particles.

The method of the invention can also generate particles which exhibit less agglomeration and generally improved handling properties. Its products tend to have smooth and relatively low energy surfaces, typically less adhesive than those of corresponding products made by prior art techniques (particularly techniques other than the Nektar™ SCF technique); they are typically in the form of free flowing powders, preferably non- or only loosely agglomerated.

When practising the method of the invention, the anti-solvent fluid must be in a compressed state, by which is meant that, at the relevant operating temperature, it is above its vapour pressure, preferably above atmospheric pressure, more preferably from 70 to 250 bar, yet more preferably from 100 to 250 bar or from 150 to 250 bar or from 180 to 220 bar. The anti-solvent fluid is preferably a fluid which is a gas at atmospheric pressure and ambient temperature. In other words, it should have a vapour pressure above 1 bar at ambient temperature (eg, at 18 to 25° C., such as at 22° C.).

More preferably “compressed” means close to, at or yet more preferably above the critical pressure P_{c} for the fluid concerned. Thus, the anti-solvent is preferably a supercritical or near-critical fluid, although it may alternatively be a compressed liquid such as for instance liquid CO₂. In practice, the pressure is likely to be in the range (1.01-9.0)P_{c}, preferably (1.01-7.0)P_{c} for a supercritical or near-critical fluid anti-solvent, or for example (0.7-3.0)P_{c}, preferably (0.7-1.7)P_{c}, for a compressed liquid anti-solvent such as liquid CO₂.

As used herein, the term “supercritical fluid” means a fluid at or above its critical pressure (P_{c}) and critical temperature (T_{c}) simultaneously. In practice, the pressure of the fluid is likely to be in the range (1.01-9.0)P_{c}, preferably (1.01-7.0)P_{c}, and its temperature in the range (1.01-4.0)T_{c} (measured in Kelvin). However, some fluids (eg, helium and neon) have particularly low critical pressures and temperatures, and may need to be used under operating conditions well in excess of (such as up to 200 times) those critical values.

“Near-critical fluid” is here used to refer to a fluid which is either (a) above its T_{c} but slightly below its P_{c}, (b) above its P_{c} but slightly below its T_{c} or (c) slightly below both its T_{c} and its P_{c}. The term “near-critical fluid” thus encompasses both high pressure liquids, which are fluids at or above their critical pressure but below (although preferably close to) their critical temperature, and dense vapours, which are fluids at or above their critical temperature but below (although preferably close to) their critical pressure.

By way of example, a high pressure liquid might have a pressure between about 1.01 and 9 times its P_{c}, and a temperature from about 0.5 to 0.99 times its T_{c}. A dense vapour might, correspondingly, have a pressure from about 0.5 to 0.99 times its P_{c}, and a temperature from about 1.01 to 4 times its T_{c}.

The terms “compressed fluid”, “supercritical fluid” and “near-critical fluid” each encompass a mixture of fluid types, so long as the overall mixture is in the compressed, supercritical or near-critical state respectively.

The anti-solvent should be a compressed (preferably supercritical or near-critical, more preferably supercritical) fluid at its point of entry into the particle formation vessel and preferably also within the vessel and throughout the particle formation process. Thus, for a carbon dioxide anti-solvent the temperature in the particle formation vessel is ideally at least 31° C., for example from 31 to 100° C., preferably from 31 to 70° C., and the pressure greater than 74 bar, for example from 75 to 350 bar, preferably from 80 to 250 bar, more preferably from 100 to 250 bar or from 180 to 220 bar.

Suitable anti-solvent back pressures, again particularly for carbon dioxide, are from 0 to 250 bar, preferably from 50 to 250 bar, more preferably from 80 to 200 bar, most preferably from 80 to 180 bar or from 100 to 150 bar.

Carbon dioxide is a highly suitable anti-solvent, but others include nitrogen, nitrous oxide, sulphur hexafluoride, xenon, ethylene, chlorotrifluoromethane and other chlorofluorocarbons, ethane, trifluoromethane and other hydrofluorocarbons, and noble gases such as helium or neon.

The anti-solvent must be miscible or substantially miscible with the fluid vehicle at the point of their contact, so that the anti-solvent can extract the vehicle from the target solution/suspension. By “miscible” is meant that the two fluids are miscible (ie, they can form a single phase mixture) in all proportions, and “substantially miscible” encompasses the situation where the fluids can mix sufficiently well, under the operating conditions used, as to achieve the same or a similar effect, ie, dissolution of the fluids in one another and precipitation of the target substance. However the anti-solvent must not, at the point of particle formation, extract or dissolve the target substance. In other words, it must be chosen so that the target substance is for all practical purposes (in particular, under the chosen operating conditions and taking into account any fluid modifiers present) insoluble or substantially insoluble in it. Preferably the target substance is less than 10⁻³ mole %, more preferably less than 10⁻⁵ mole %, soluble in the anti-solvent fluid.

The anti-solvent fluid may optionally contain one or more modifiers, for example water, methanol, ethanol, isopropanol or acetone. A modifier (or co-solvent) may be described as a chemical which, when added to a compressed fluid such as a supercritical or near-critical fluid, changes the ability of that fluid to dissolve other materials. When used, a modifier preferably constitutes not more than 40 mole %, more preferably not more than 20 mole %, and most preferably from 1 to 10 mole %, of the anti-solvent fluid.

The vehicle is a fluid which.is able to carry the target substance in solution or suspension. It may be composed of one or more component fluids, eg, it may be a mixture of two or more solvents. It must be soluble (or substantially soluble) in the chosen anti-solvent fluid at their point of contact. It may contain, in solution or suspension, other materials apart from the target substance.

The target solution/suspension may in particular comprise two or more fluids which are mixed in situ at or immediately before their point of contact with the anti-solvent. Such systems are described, eg, in WO-96/00610 and WO-01/03821. The two or more fluids may carry two or more target substances, to be combined in some way (for instance, co-precipitated as a matrix, or one precipitated as a coating around the other, or precipitated as the product of an in situ reaction between the substances) at the point of particle formation. Target substance(s) may also be carried in the anti-solvent fluid as well as in the target solution(s)/suspension(s).

A target substance may be any substance which needs to be produced in particulate form. Examples include pharmaceuticals; nutraceuticals; pharmaceutical or nutraceutical excipients such as carriers; dyestuffs; cosmetics; foodstuffs; coatings; agrochemicals; products of use in the ceramics, explosives or photographic industries; etc. It may be organic or inorganic, monomeric or polymeric. It is preferably soluble or substantially soluble in the relevant fluid vehicle, preferably having a solubility in it of 10⁻⁴ mole % or greater under the conditions under which the target solution is prepared (ie, upstream of the point of particle formation).

In a preferred embodiment of the invention, the target substance is for use in or as a pharmaceutical or pharmaceutical excipient. It may in particular comprise a pharmaceutically active substance, especially one for which a small particle size and/or narrow particle size distribution is important, for instance a drug intended for use in inhalation therapy or one for which rapid and/or efficient in vivo dissolution is desirable. The present invention is also highly suitable for producing target substances for which a high degree of purity (including polymorphic purity and/or reduced residual solvent levels) is desired.

The target substance may be in a single or multi-component form (eg, it could comprise an intimate mixture of two materials, or one material in a matrix of another, or one material coated onto a substrate of another, or other similar mixtures). The particulate product, formed from the target substance(s) using the method of the invention, may also be in such a multi-component form—examples include two pharmaceuticals intended for co-administration, or a pharmaceutical together with a polymer carrier matrix. Such products may be made, as described above, from solutions/suspensions containing only single component starting materials, provided the solutions/suspensions are contacted with the anti-solvent fluid in the correct manner. The particulate product may comprise a substance formed from an in situ reaction (ie, immediately prior to, or on, contact with the anti-solvent) between two or more reactant substances each carried by an appropriate fluid.

Particular examples of coated multi-component products include where one target substance is an active (eg, pharmaceutically active) substance, and another an excipient to be deposited as a coating around the first one (for example to provide a controlled release and/or taste-masked drug formulation). Alternatively one target substance may comprise a core of an excipient onto which an active substance is to be coated. A yet further alternative is that both target substances are active substances, for instance pharmaceutically active materials intended for co-administration.

In the method of the invention, the anti-solvent and the target solution/suspension are introduced separately into the particle formation vessel (which is preferably the vessel in which the formed particles are collected) and contact each other after (preferably immediately after) the point of anti-solvent entry into the vessel. Their contact should however take place at the same or substantially the same point as the target solution/suspension enters the vessel, as a consequence of the solution/suspension being introduced directly into the anti-solvent flow. In this way, particle formation can be made to occur at a point where there is a high degree of control over conditions such as the temperatures, pressures and flow rates of the fluids.

The fluids are ideally introduced in such a way that the mechanical (kinetic) energy of the anti-solvent fluid can act to disperse the target solution/suspension at the same time as it extracts the vehicle; this again allows a high degree of control over the physicochemical characteristics of the particulate product, in particular the size and size distribution of the particles and their solid state properties. “Disperse” in this context refers generally to the transfer of kinetic energy from one fluid to another, usually implying the formation of droplets, or of other analogous fluid elements, of the fluid to which the kinetic energy is transferred. Thus, the first and second fluid inlets should be so constructed and arranged that they allow the mechanical energy (typically the shearing action) of the anti-solvent flow to facilitate intimate mixing of the fluids and to disperse them, at the point where the fluids meet.

Introducing the two fluids separately can help prevent apparatus blockages at their points of entry (in particular at the anti-solvent inlet), due for example to the highly efficient extraction of the vehicle into the anti-solvent under the operating conditions used. At the same time, the special form of fluid inlet provided in accordance with the invention can help to retain a high degree of control over the mechanism for fluid contact, by introducing the target solution/suspension directly into the anti-solvent flow within the particle formation vessel.

Such control may if desired be improved upon by providing controlled agitation within the vessel, and/or within one or more of the fluid inlets, in particular in the region of fluid contact immediately downstream of the respective target solution/suspension and anti-solvent inlets. For example, the target solution/suspension may be dispersed onto a sonicating surface at or immediately prior to its contact with the anti-solvent fluid. Agitation may alternatively be achieved for instance by stirring, such as with a turbine, propeller, paddle, impeller or the like.

That said, the present invention may if necessary be practised in the absence of such additional agitation means, particularly within the particle formation vessel.

Generally it is unnecessary in the method of the present invention to use more than one anti-solvent flow stream in order to disperse, and extract the vehicle from, the target solution/suspension. Thus, an impinging flow of a second anti-solvent fluid, such as is used to aid dispersion in the Nektar™ SCF process described in WO-98/36825, is typically not present when practising the method of the present invention.

The target solution/suspension may be introduced into the vessel through any suitable fluid inlet, including one which effects, or assists in effecting, controlled atomisation of the solution/suspension. The anti-solvent fluid is preferably introduced through a nozzle, more preferably a convergent nozzle which focuses a flowing fluid stream through a smaller area outlet.

The target solution/suspension may be introduced with a back pressure, for instance from 5 to 250 bar or from 50 to 200 bar or from 50 to 150 bar. This can generally be achieved by manipulation of the solution/suspension flow rate, the pressure in the particle formation vessel and the size and geometry of the outlet through which it is introduced into the vessel. In particular, the outlet may present a restriction in the solution/suspension flow as it enters the vessel, as for example in a nozzle or other similar reduced area outlet.

Preferably the target solution/suspension and the anti-solvent meet immediately downstream of the point of anti-solvent entry. “Immediately” in this context implies a sufficiently small time interval (between the anti-solvent entering the particle formation vessel and its contact with the target solution/suspension) as preferably still to allow transfer of mechanical energy from the anti-solvent to the solution/suspension so as to achieve dispersion. Nevertheless, there is still preferably a short interval of time between anti-solvent entry and fluid contact so as to eliminate, or substantially eliminate or at least reduce, the risk of apparatus blockage due to particle formation at the points of fluid (in particular anti-solvent) entry. The timing of the fluid contact will depend on the natures of the fluids, the target substance and the desired end product, as well as on the size and geometry of the particle formation vessel and the fluid inlets and on the fluid flow rates. The contact may occur within 0 or 0.001 to 25 or 50 milliseconds, preferably within 0.001 to 10 milliseconds, more preferably within 0.01 to 5 or 10 milliseconds, of the anti-solvent entering the particle formation vessel. Generally it will occur within 0.1 milliseconds, preferably within 0.05 or 0.02 milliseconds, more preferably within 0.01 milliseconds, still more preferably within 0.005 or even 0.001 milliseconds, of the anti-solvent entering the vessel.

The target solution/suspension is typically introduced directly into the anti-solvent flow, and thus meets with the anti-solvent flow at the point where the target solution/suspension enters the vessel. Thus, where for instance the anti-solvent inlet generates a conical fluid stream, the outlet of the first fluid inlet should be located, in use, within the emerging anti-solvent cone—the dimensions of this cone depend, inter alia, on the geometry of the anti-solvent outlet, on the anti-solvent velocity and also on the operating conditions (eg, temperature, pressure and anti-solvent back pressure). The diameter of the cone will also increase with distance from the anti-solvent outlet.

The outlet of the first fluid inlet should be located on or close to the main (central) axis of anti-solvent flow, suitably within 2 mm, more preferably within 1 mm and most preferably within 0.5 or 0.3 mm of that axis (these distances being measured in a plane perpendicular to the axis itself). Typically this means that the solution/suspension outlet is in line with, or close to being in line with, the central longitudinal axis of the second fluid inlet.

Again the degree of separation which is tolerable between the target solution/suspension outlet and the axis of anti-solvent flow may depend on factors such as those listed above which determine the geometry of the anti-solvent flow stream, it being generally desirable for the target solution/suspension to be introduced directly into the anti-solvent flow and also at a location where the anti-solvent has sufficient kinetic energy to achieve efficient dispersion of the target solution/suspension.

It may be suitable for the separation between the target solution/suspension outlet and the main axis of anti-solvent flow (measured in a plane perpendicular to that axis) to be no more than 10 times, preferably no more than 8 times, more preferably no more than 5 times or 3 times the diameter of the anti-solvent outlet.

Preferably the outlet of the first fluid inlet is located vertically below that of the second fluid inlet, and the anti-solvent fluid flows into the particle formation vessel in a vertically downwards direction. “Vertical” for these purposes includes directions which are no more than 30 or preferably 20 or 10° from the vertical.

At the point where the target solution/suspension and the anti-solvent meet, the angle between their axes of flow may be from 10° (ie, the two fluids are flowing in almost parallel directions) to 180° (ie, oppositely-directed flows), more typically from 45 to 135°. However, they preferably meet at a point where they are flowing in approximately perpendicular directions, ie, the angle between their axes of flow is from 70 to 110°, more preferably from 80 to 100°, such as 90°.

Suitable fluid inlet means, which may be used to achieve the form of fluid contact required by the present invention, are described in more detail below.

Use of such a fluid inlet system can allow GAS-based particle formation techniques to be practised in cases where the vehicle for the target solution/suspension is a relatively high boiling fluid (eg, boiling point greater than about 150° C., or even greater than 180° C.) such as dimethyl formamide (DMF), dimethyl sulphoxide (DMSO), dimethyl acetamide (DMA), diethyl acetamide (DEA) or N-methyl pyrollidinone (NMP), or where the target substance is temperature sensitive. Since the anti-solvent and the target solution/suspension enter the vessel separately, the latter can be maintained at a desired lower temperature despite the use of a relatively high temperature for the incoming anti-solvent.

When carrying out the present invention, the particle formation vessel temperature and pressure are ideally controlled so as to allow particle formation to occur at the same or substantially the same point as the target solution/suspension meets the anti-solvent fluid (which is generally the point at which the target solution/suspension enters the vessel). The conditions in the vessel must generally be such that the anti-solvent fluid, and the solution which is formed when it extracts the vehicle, both remain in the compressed (preferably supercritical or near-critical, more preferably supercritical) form whilst in the vessel. For the supercritical, near-critical or compressed solution, this means that at least one of its constituent fluids (usually the anti-solvent fluid, which in general will be the major constituent of the mixture) should be in a supercritical, near-critical or compressed state, as the case may be, at the time of particle formation. There should at that time be a single-phase mixture of the vehicle and the anti-solvent fluid, otherwise the particulate product might be distributed between two or more fluid phases, in some of which it might be able to redissolve. This is why the anti-solvent fluid needs to be miscible or substantially miscible with the vehicle.

The terms “supercritical solution”, “near-critical solution” and “compressed solution” mean respectively a supercritical, near-critical or compressed fluid together with a fluid vehicle which it has extracted and dissolved. The solution should itself still be in the supercritical, near-critical or compressed state, as the case may be, and exist as a single phase, at least within the particle formation vessel.

Selection of appropriate operating conditions will be influenced by the natures of the fluids involved (in particular, their P_{c} and T_{c} values and their solubility and miscibility characteristics) and also by the characteristics desired of the particulate end product, for instance yield, particle size and size distribution, purity, morphology or crystalline, polymorphic or isomeric form. Variables include the flow rates of the anti-solvent fluid and the target solution/suspension, the concentration of the target substance in the vehicle, the temperature and pressure inside the particle formation vessel, the anti-solvent temperature upstream of the vessel and the construction and relative positioning of the fluid inlets into the vessel, in particular the sizes of the target solution/suspension and anti-solvent outlets and the distance between them. The method of the invention preferably involves controlling one or more of these variables so as to influence the physicochemical characteristics of the particles formed.

The flow rate of the anti-solvent fluid relative to that of the target solution/suspension, and its pressure and temperature, should be sufficient to allow it to accommodate the vehicle, so that it can extract the vehicle and hence cause particle formation. The anti-solvent flow rate will generally be higher than that of the target solution/suspension—typically, the ratio of the target solution/suspension flow rate to the anti-solvent flow rate (both measured as volumetric flow rates, at or immediately prior to the two fluids coming into contact with one another) will be 0.001 or greater, preferably from 0.005 to 0.2, more preferably from 0.01 to 0.2, yet more preferably from 0.01 to 0.1, such as from 0.03 to 0.1.

The anti-solvent flow rate will also generally be chosen to ensure an excess of the anti-solvent over the vehicle when the fluids come into contact, to minimise the risk of the vehicle re-dissolving and/or agglomerating the particles formed. At the point of its extraction into the anti-solvent, the vehicle may constitute from 0.5 or 1 to 80 mole %, preferably 50 mole % or less or 30 mole % or less, more preferably from 1 to 20 mole % and most preferably from 1 to 10 or from 1 to 5 mole %, of the compressed fluid mixture formed.

Both the anti-solvent and the target solution/suspension are ideally introduced into the particle formation vessel with a smooth, continuous and preferably pulse-less or substantially pulse-less flow. Conventional apparatus may be used to ensure such fluid flows.

The method of the invention preferably additionally involves collecting the particles following their formation, more preferably in the particle formation vessel itself.

Apparatus suitable for use in carrying out the method of the invention preferably comprises:
(i) a particle formation vessel;
(ii) a first fluid inlet for introducing a target solution/suspension into the vessel; and
(iii) a second fluid inlet, separate from the first, for introducing a compressed fluid anti-solvent into the vessel;
   wherein the outlet of the first fluid inlet is downstream (in the direction of anti-solvent flow in use) of, and in line with, that of the second fluid inlet. By “in line with” is meant that the outlet of the first fluid inlet lies on or close to the main (central) axis of anti-solvent flow out of the second fluid inlet, as described above. The outlet of the first fluid inlet is preferably immediately downstream of that of the second fluid inlet.

Thus, the first fluid inlet should be positioned such that, in use, its outlet is within the flow of anti-solvent exiting the second fluid inlet. Most preferred is an arrangement in which the centre of the outlet of the first fluid inlet corresponds to the centre of the outlet of the second fluid inlet, ie, the centres of the two outlets are both positioned along the main axis of anti-solvent flow, typically also in line with the central longitudinal axis of the second fluid inlet.

The first fluid inlet suitably comprises a fluid inlet tube, for instance of stainless steel or fused silica, which might typically have an internal diameter of from 0.1 to 0.2 mm, more preferably from 0.1 to 0.15 mm, and may have a tapered outlet section. In some embodiments, the first fluid inlet may have an outlet of diameter less than 0.1 mm, preferably of 0.09 or 0.08 mm or less, more preferably of 0.07 or 0.06 mm or less, yet more preferably of 0.05 or 0.04 or 0.03 mm or less. Its outlet diameter may for instance be in the range 0.02 to 0.08 mm, or from 0.02 to 0.07 mm. In cases its outlet diameter may be as low as 0.01 or even 0.005 mm.

The first fluid inlet may comprise an outlet tube section, for example a length of capillary tubing, having an outlet diameter of for instance less than 0.1 mm, mounted in fluid communication with a wider diameter tube section through which fluid may be directed from its source towards the outlet tube section and thence to the particle formation vessel. The resultant reduction in tube diameter may be used to induce a back pressure in the target solution/suspension flow, the magnitude of which may be varied by altering for example the length and/or outlet diameter of the outlet tube section.

The second fluid inlet preferably provides a restriction at the point of fluid entry into the particle formation vessel: for instance, the second fluid inlet may comprise a nozzle. Again it may suitably be made from stainless steel. It preferably has at least one passage of internal diameter from for instance 1 to 2 mm, more preferably from 1.3 to 1.9 mm, such as 1.6 mm. Again, it may have a tapered outlet section (ie, be a “convergent”-type nozzle), with an angle of taper (with respect to the central longitudinal axis of the nozzle) typically in the range 10 □ to 70 □, such as from 20 □ to 40 □ or from 50 to 70 □. Alternatively it may have a divergent outlet, with the same typical angle of taper as for the convergent version. A nozzle of the convergent-divergent type may also be suitable for use as the second fluid inlet.

The opening at the outlet end (tip) of the second fluid inlet will preferably have a diameter in the range of 0.005 to 5 mm, more preferably 0.05 to 2 mm, most preferably from 0.1 to 0.5 mm, for instance about 0.1, 0.2, 0.3 or 0.35 mm. In cases a smaller diameter outlet may be preferred as it may contribute to higher yields; an outlet diameter of from 0.1 to 0.3 mm or from 0.15 to 0.25 mm may therefore be suitable for the second fluid inlet.

The dimensions of the fluid inlets will naturally depend on the scale on which the process is to be practised; for commercial scale manufacture, for example, the above nozzle dimensions may be up to ten times larger.

A nozzle of the above type may comprise more than one fluid passage; for instance it may comprise two or more coaxial passages such as in the nozzles described in WO-95/01221, WO-96/00610 and WO-98/36825, particularly if additional fluids are to be introduced into the system. One or more of the passages may be used to introduce two or more fluids at the same time, and the inlets to such passages may be modified accordingly.

A suitable separation for the outlets of the first and second fluid inlets is a short distance such as from 0 or 0.1 to 50 times, preferably from 10 to 40 times, more preferably from 10 to 30 or from 15 to 25 times, the diameter of the outlet of the second fluid inlet. In some cases the preferred separation might be from 15 to 20 times the diameter of the outlet of the second fluid inlet, in others from 18 to 27 or from 20 to 25 times. Suitable distances might lie from 0 to 10 mm or from 0.1 or 0.5 to 10 mm, preferably from 2 to 8 mm or from 2 to 6 mm, for instance about 4 or 5 mm, in particular for an anti-solvent outlet diameter of 0.2 mm or thereabouts. For an anti-solvent outlet diameter of 0.4 mm or thereabouts, a suitable distance between the two fluid outlets might be from 3 or 4 to 10 mm or from 6 to 8 mm, such as about 7 mm. Where a smaller diameter target solution/suspension inlet is used, smaller separations may be appropriate, such as from 0 to 2 mm, preferably from 0 or 0.1 mm to 1.5 or 1 mm.

What constitutes a separation of “0” may depend on practical constraints such as the thickness of the walls of the inlets and the assembly in which they are mounted; generally it will correspond to the two outlets being as close to coincident as possible. Again, the separation between outlets may depend on the scale of the process which the inlets are to be used for.

It is believed, although we do not wish to be bound by this theory, that there may be an optimum separation between the two outlets which represents a balance between avoiding undue agglomeration of the particles as they form whilst also maximising the efficiency of fluid mixing and vehicle extraction. If the target solution/suspension is introduced into the anti-solvent flow close to the outlet of the second fluid inlet, then fluid mixing will be highly efficient and particle formation rapid, but there may also be an increased tendency for agglomeration of the particles as they form, resulting ultimately in a larger diameter product. Conversely, if the two fluids meet at too great a distance from the outlet of the second fluid inlet, then fluid mixing and vehicle extraction may be less efficient, reducing control over the product characteristics (including particle size and morphology), potentially allowing more particle growth and higher residual solvent levels and possibly also reducing yields.

Such distances are suitably measured between the centres of the relevant fluid outlets, or alternatively (in particular for a smaller diameter fluid outlet) from the external wall of for instance a fluid inlet tube, suitably from that point on the external wall which is closest to the anti-solvent outlet.

The outlet of the first fluid inlet preferably has a smaller cross sectional area than that of the second fluid inlet, more preferably less than 80% as large and most preferably less than 70% or even 50% as large. In some cases the cross sectional area of the target solution/suspension outlet may be less than 50% as large, more preferably less than 30% or 25% or 20% as large and most preferably less than 10% or 8% or 5% or 3% as large, as that of the second fluid inlet. In cases it may be less than 2% or 1% or 0.5% or 0.1% as large as that of the second fluid inlet.

The first and second fluid inlets are preferably arranged so that at the point where the target solution/suspension and the anti-solvent meet, the angle between their axes of flow is from 70 to 110°, more preferably from 80 to 100°, most preferably about 90°.

The first and second fluid inlets may for convenience be provided as part of a single fluid inlet assembly which may be placed in fluid communication with the particle formation vessel and with sources of the anti-solvent fluid and the target solution/suspension.

The particle formation vessel preferably contains particle collection means, such as a filter, by which particles of the target substance may be collected in the vessel in which they form, downstream of the point of contact between the target solution/suspension and the anti-solvent fluid.

The apparatus may additionally comprise a source of a compressed (preferably supercritical or near-critical) fluid and/or a source of a target solution or suspension. The former may itself comprise means for altering the temperature and/or pressure of a fluid so as to bring it into a compressed (preferably supercritical or near-critical) state.

The apparatus conveniently includes means for controlling the pressure in the particle formation vessel, for example a back pressure regulator downstream of the vessel, and/or means (such as an oven) for controlling the temperature in the vessel. The vessel is conveniently a pressure vessel and should be capable of withstanding the pressures necessary to maintain compressed (preferably supercritical or near-critical) conditions during the particle formation process.

A second aspect of the present invention provides a particulate product formed using a method according to the first aspect.

Because embodiments of the present invention are modified versions of the inventions disclosed in WO-95/01221, WO-96/00610, WO-98/36825, WO-99/44733, WO-99/59710, WO-01/03821, WO-01/15664, WO-02/38127 and WO-03/008082, technical features described in those documents, for instance regarding the selection of appropriate reagents and operating conditions, can apply also to the present invention. The nine earlier documents are therefore intended to be read together with the present application.

In this specification the term “substantially”, when applied to a condition, is meant to encompass the exact condition (eg, exact simultaneity) as well as conditions which are (for practical purposes, taking into account the degree of precision with which such conditions can be measured and achieved) close to that exact condition, and/or which are similar enough to that exact condition as to achieve, in context, the same or a very similar effect.

References to solubilities and miscibilities, unless otherwise stated, are to the relevant fluid characteristics under the operating conditions used, ie, under the chosen conditions of temperature and pressure and taking into account any modifiers present in the fluids.

The present invention will now be illustrated with reference to the following non-limiting examples and the accompanying figures, of which:

FIG. 1 is a plot of the enthalpy variation of CO₂ with temperature and pressure, illustrating the change in CO₂ temperature during its isenthalpic expansion;

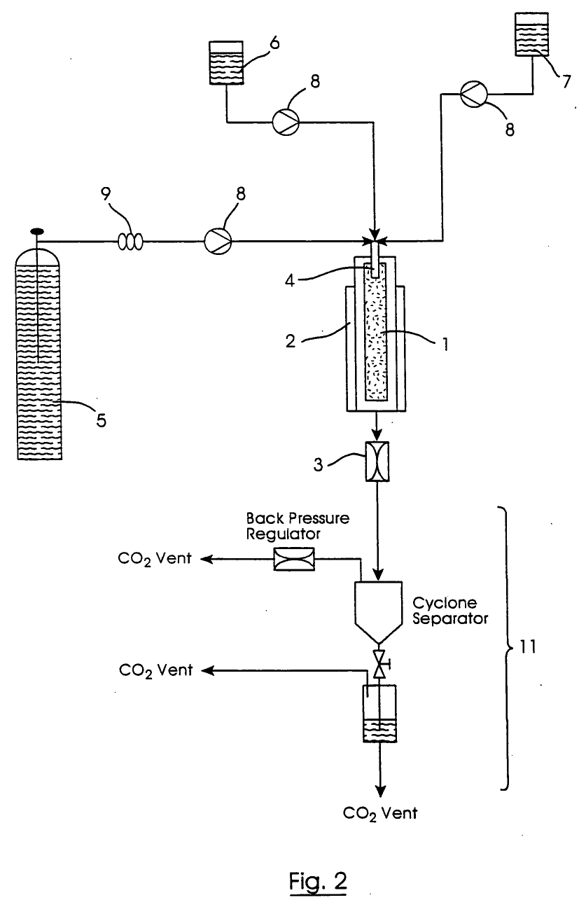

FIG. 2 illustrates schematically apparatus suitable for use in carrying out a method according to the present invention;

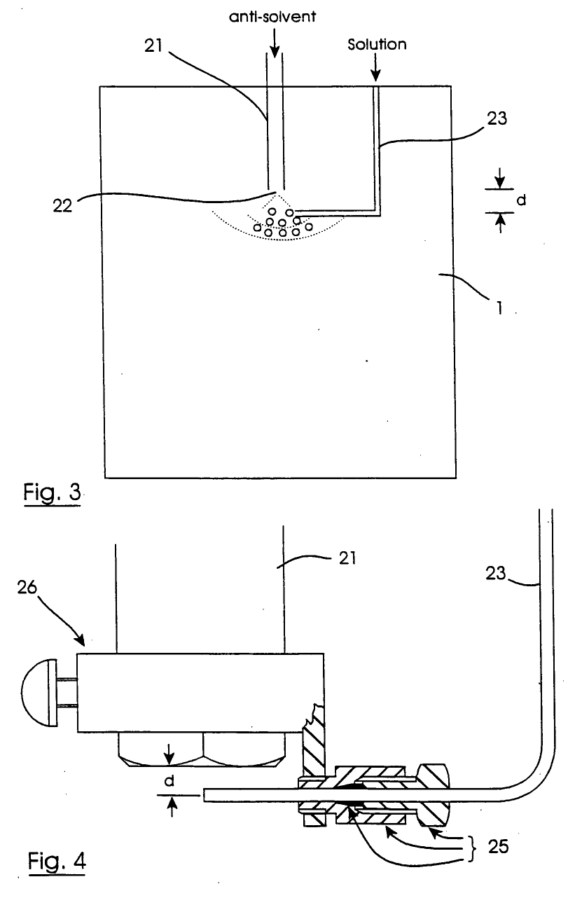

FIGS. 3 to 5 are schematic longitudinal cross sections and an under plan view respectively of parts of a fluid inlet assembly useable with the FIG. 2 apparatus;

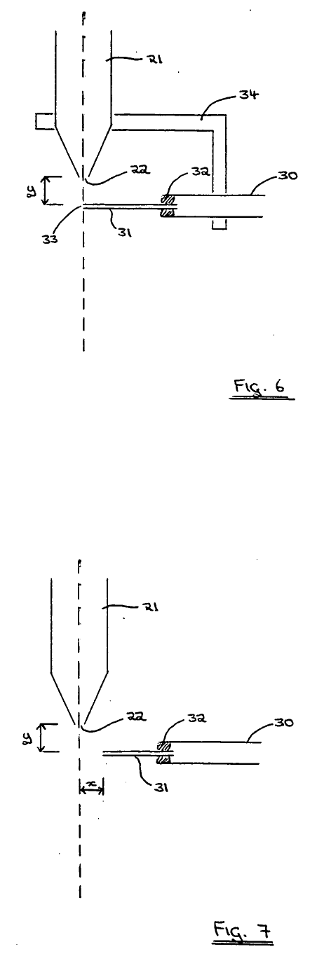

FIGS. 6 and 7 are schematic longitudinal cross sections through parts of an alternative fluid inlet assembly useable with the FIG. 2 apparatus;

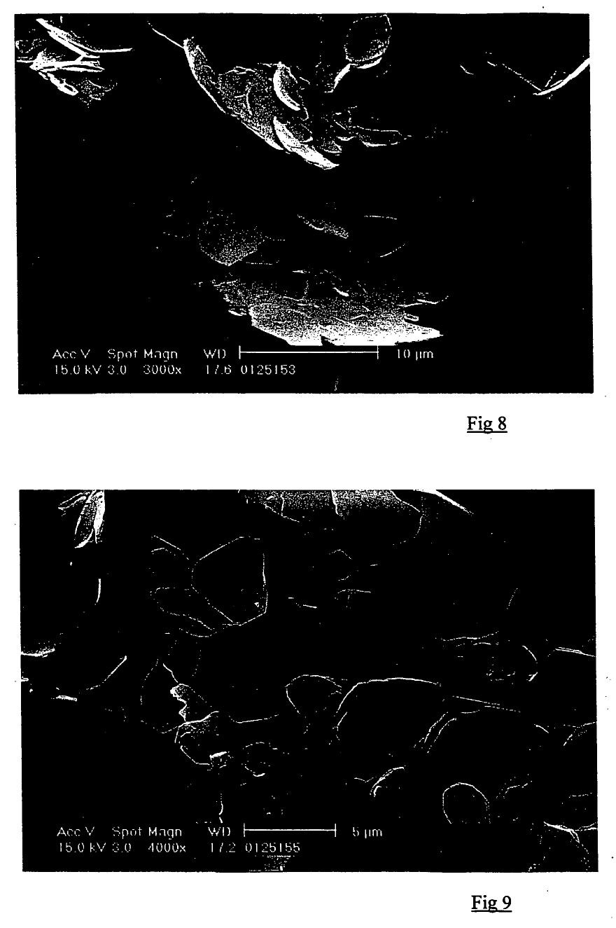

FIGS. 8 and 9 are SEMs (scanning electron micrographs) of the respective products of Examples A3 and A4 below; and

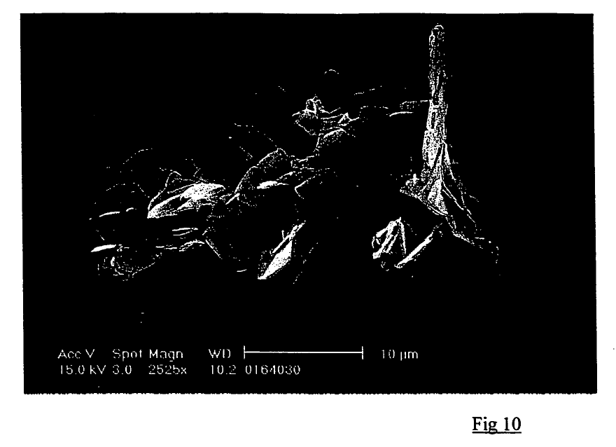

FIG. 10 is an SEM of the product of Example B1 below.

### DETAILED DESCRIPTION

FIG. 2 shows apparatus suitable for carrying out methods in accordance with the present invention. Item 1 is a particle formation vessel, within which the temperature and pressure can be controlled by means of the heating jacket 2 and back pressure regulator 3. The vessel 1 contains a particle collection device (not shown) such as a filter, filter basket or filter bag. A fluid inlet assembly 4 allows introduction of a compressed (typically supercritical or near-critical) fluid anti-solvent from source 5 and one or more target solutions/suspensions (or additional fluid vehicles if desired) from sources such as 6 and 7. The items labelled 8 are pumps, and 9 is a cooler. A recycling system 11 allows vehicle recovery.

The fluid inlet assembly 4 may for example take the form shown in FIGS. 3 to 5. FIG. 3 shows the assembly schematically, in use with the particle formation vessel 1 of the FIG. 2 apparatus. Nozzle 21 is for introduction of the anti-solvent fluid. It has only a single passage of circular cross section, with a circular outlet 22. Alternatively, a multi-component nozzle may be used, with anti-solvent introduced through one or more of its passages and the remaining passages either closed off or else used to introduce additional reagents. (For example, a multi-passage nozzle of the type described in WO-95/01221 or WO-96/00610 may be used. Such nozzles have two or more concentric (coaxial) passages, the outlets of which are typically separated by a short distance to allow a small degree of internal mixing to take place between fluids introduced through the respective passages before they exit the nozzle. The anti-solvent could for instance be introduced through the inner passage of such a nozzle, traversing a small “mixing” zone as it exits that inner passage and then passing through the main nozzle outlet into the particle formation vessel.)

Although this is not shown in FIG. 3, the nozzle 21 may have a tapered (typically convergent) tip, with an outlet of smaller diameter than that of the main nozzle passage.

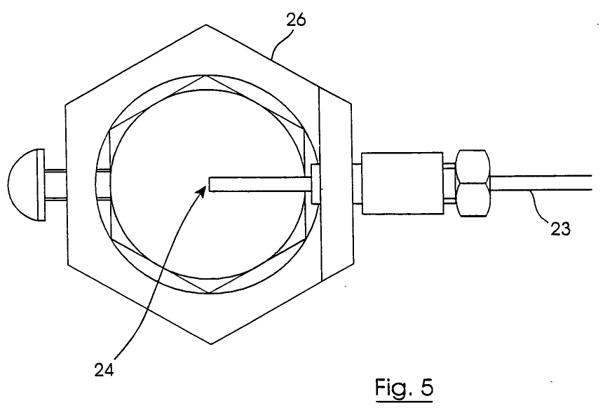

Inlet tube 23 is for introduction of the target solution/suspension, and is so shaped and located that the direction of flow of the solution/suspension at its outlet 24 (see FIG. 5) will be perpendicular to that of the anti-solvent exiting nozzle 21. Again the tube is of circular cross section.

FIG. 4 shows how tube 23 is mounted, by means of the supporting and locking pieces 25, on a collar 26 which is itself mounted around the lower portion of the nozzle 21. The arrangement is such as to allow adjustment of the distance “d” between the outlets of nozzle 21 and tube 23. It can be seen that the outlet of tube 23 is positioned in line with the central longitudinal axis of the nozzle 21.

Both the nozzle 21 and the tube 23 are preferably made from stainless steel.

The assembly of FIGS. 3 to 5 may be less likely to suffer blockages (at the nozzle and tube outlets) than when a multi-component nozzle of the type described in WO-95/01221 is used to co-introduce the anti-solvent and target solution/suspension together, particularly when the operating conditions are such as to allow a very rapid and efficient removal of the solvent vehicle, from the target solution/suspension, by the anti-solvent.

An alternative fluid inlet assembly 4, for use in the FIG. 2 apparatus, is illustrated in FIGS. 6 and 7. Again a nozzle 21, as in FIGS. 3 to 5, is used to introduce the anti-solvent via its outlet 22, which again is preferably tapered. A tube 30, similar to tube 23 in FIGS. 3 to 5, introduces the target solution/suspension.

A length of thin capillary tubing 31 is mounted, preferably although not necessarily centrally, within the outlet of tube 30, for instance with a suitable adhesive 32. This gives a much smaller (eg, of the order of 0.05 mm) effective diameter for the solution outlet 33. The length of the capillary 31, and its cross-sectional area relative to that of the tube 30, determine the degree of back pressure generated in the target solution flow on entering the vessel 1.

Other ways of achieving a small solution outlet are of course possible, for instance using an alternative form of connection between the main solution inlet tube 30 and a smaller diameter outlet tube section.

The tube 30 and capillary 31 are preferably fixed in position relative to one another. They are mounted in a support (shown schematically at 34) which allows their horizontal (x) and vertical (y) separations from the anti-solvent nozzle outlet 22 to be varied, preferably continuously, for instance as described below in connection with Examples A. FIG. 6 for instance shows the solution outlet 33 directly in line with the central longitudinal axis of the nozzle 21 (ie, with the main axis of anti-solvent flow) and FIG. 7 shows the outlet 33 displaced from that axis by a distance x, shown exaggerated for clarity.

The relative positions of the anti-solvent and solution outlets are preferably similarly variable in the fluid inlet assembly of FIGS. 3 to 5.

### EXAMPLES A

Apparatus as shown in FIG. 2, incorporating a fluid inlet assembly as shown in FIGS. 3 to 5, was used to carry out particle formation methods in accordance with the invention.

The nozzle 21 comprised a fluid inlet tube of internal diameter 0.75 mm, a convergent tip with a 60° half angle taper (with respect to the central longitudinal nozzle axis) and an outlet of diameter 0.2 mm. According to theory, this generates a fluid jet with a cone angle of approximately 20°.

The internal bore at the end of the inlet tube 23 was 0.125 mm.

The experiments investigated the effect of varying both (a) the horizontal distance x between the solution line outlet and the central axis of anti-solvent flow, and (b) the vertical distance y between the nozzle outlet 22 and the solution line outlet (y being measured, for convenience, from the top external wall of the solution inlet tube 23).

Supercritical carbon dioxide, pre-heated to 70° C., was used as the anti-solvent. It was pumped at a flow rate (of liquid CO₂, measured at the pump head) of 200 ml/min.

The target solution contained 3% w/v salmeterol xinafoate in methanol, and was introduced at a flow rate of 4 ml/min.

The pressure in the particle formation vessel 1 (capacity 2 litres) was maintained at 200 bar, the temperature at 333 K (60° C.). The CO₂ velocity at the nozzle outlet 22 was sub-sonic throughout the experiments.

Particle formation was allowed to occur by the action of the CO₂ anti-solvent, and the products collected in the vessel 1. The run time of each experiment was 50 minutes, corresponding to 6 g of salmeterol xinafoate being processed. The products were assessed by scanning electron microscopy (SEM) and their particle sizes analysed using a Sympatec™ apparatus at 2 bar shear pressure.

The results are shown in Table 1. In practice, a “0” value for y represents as close to zero as was possible without cutting into the nozzle or inlet tube walls.

TABLE 1

| | | | Yield | Particle | Particle |
|---|---|---|---|---|---|
| Exp^{t}. | | | (% | size | size |
| no. | x (mm) | y (mm) | w/w) | (μm)* | spread** |
| A1 | 0 | 0 | 83 | 10.2 | 2.12 |
| A2 | 0 | 2 | 85 | 9.6 | 2.06 |
| A3 | 0 | 4 | 84 | 8.6 | 2.04 |
| A4 | 0 | 6 | 80 | 8.2 | 2.05 |
| A5 | 0 | 8 | 83 | 8.9 | 2.07 |
| A6 | 2 | 0 | 76 | 9.5 | 2.16 |
| A7 | 2 | 4 | 73 | 9.0 | 2.08 |
| A8 | 2 | 8 | 83 | 9.1 | 2.16 |
| A9 | 4 | 8 | 79 | 9.0 | 2.11 |

*Volume mean diameter, representing the average of two analyses (average error approximately 0.2 μm).
**Particle size spread is defined as (D₉₀ − D₁₀)/D₅₀ where D is the volume mean diameter of the relevant particle population.

SEMs of the products of Examples A3 and A4 are shown in FIGS. 8 and 9 respectively.

These data demonstrate that improved yields, smaller particle sizes and tighter size distributions can generally be achieved by locating the target solution outlet directly in line with the main axis of anti-solvent flow (x=0)—compare for instance Example A5 with A8, and A3 with A7.

They also show that improvements in particle size and distribution can generally be achieved, in this case, by locating the target solution outlet between 4 and 8 mm, preferably between 4 and 6 mm, from the anti-solvent nozzle outlet (y=4-8 mm)—compare for instance Examples A3 to A5 with Examples A1 and A2, and Examples A7 and A8 with Example A6. In these experiments the preferred vertical separation y, of 4-6 mm, was between 20 and 30 times the nozzle outlet diameter.

If the target solution and anti-solvent outlets are closer together (y=0 for example), then particle sizes appear to increase, this possibly being due to increased agglomeration.

### EXAMPLES B

Apparatus as shown in FIG. 2, incorporating a fluid inlet assembly as shown in FIGS. 6 and 7, was used to carry out a further particle formation method in accordance with the invention. The nozzle 21 was the same as used in Examples A.

The target solution inlet comprised a fused silica capillary of length 20 mm and internal diameter 50 μm, glued into a standard 1.59 mm (1/16″) internal diameter stainless steel tube. Its outlet, into the particle formation vessel 1, was therefore 50 μm in diameter, and its cross sectional area only 6% of that of the outlet of nozzle 21. A two-component epoxy resin was used to secure the capillary in place, under elevated temperatures (180° C.) to enhance the mechanical strength of the bond. Due to the viscous flow of the uncured resin, it was not possible to centre the capillary within the stainless steel tube.

The vertical separation “y” was ˜0.5-1 mm.

Again supercritical carbon dioxide was used as the anti-solvent, pumped at a flow rate (of liquid CO₂, measured at the pump head) of 200 ml/min, and the target solution contained 3% w/v salmeterol xinafoate in methanol, introduced at a flow rate of 4 ml/min. The back pressure measured across the solution inlet was 85 bar.

The particle formation vessel capacity was 2 litres. The pressure in the vessel was 200 bar, the temperature 333 K (60° C.). The CO₂ velocity at the nozzle outlet 22 was sub-sonic.

As in Examples A, particle formation occurred by the action of the CO₂ anti-solvent, and the product was collected in the vessel 1. The product was assessed by scanning electron microscopy (SEM) and its particle size analysed using a Sympatec™ apparatus at 2 bar shear pressure.

Table 2 shows the results, and FIG. 10 an SEM of the product.

TABLE 2

| | Yield | Particle | | | | Particle |
|---|---|---|---|---|---|---|
| Exp^{t}. | (% | size | D₁₀ | D₅₀ | D₉₀ | size |
| no. | w/w) | (μm) | (μm) | (μm) | (μm) | spread |
| B1 | 73 | 8.23 | 1.86 | 6.96 | 16.39 | 2.09 |

This experiment illustrates that fine particles, with a narrow size distribution, can be successfully produced using the method of the invention with a much smaller target solution inlet and higher solution back pressure.

### COMPARATIVE EXAMPLES C

The results of Examples A, carried out in accordance with the present invention, were compared with those obtained from Examples C, in which a two-component coaxial nozzle, of the type shown in FIG. 3 of WO-95/01221, was used to co-introduce a 3% w/v salmeterol xinafoate in methanol solution and a supercritical CO₂ anti-solvent.

The operating temperature and pressure, within the particle formation vessel, were as for Examples A, ie, 60° C. and 200 bar. The CO₂ flow rate was 200 ml/min (ie, sub-sonic velocity), the target solution flow rate 4 ml/min. The nozzle used had a convergent tip (60° half angle) with either a 0.2 mm or a 0.4 mm outlet diameter. The target solution was introduced through the outer nozzle passage (internal diameter 2.3 mm) and the anti-solvent through the inner passage (internal diameter 0.75 mm). The particle formation vessel had a capacity of 2 litres.

The results are shown in Table 3, which also shows (for ease of comparison) the results of Examples A3 to A5 which represent preferred modes of practising the present invention.

TABLE 3

| | | | Nozzle outlet | Yield | | |
|---|---|---|---|---|---|---|
| | x | y | diameter | (% | Particle size | Particle size |
| Ex. | (mm) | (mm) | (mm) | w/w) | (μm)* | spread |
| C1 | — | — | 0.4 | 84 | 8.6 ± 0.2 | 2.26 |
| C2 | — | — | 0.2 | 73 | 9.9 ± 0.2 | 2.15 |
| A3 | 0 | 4 | 0.2 | 84 | 8.6 ± 0.1 | 2.04 |
| A4 | 0 | 6 | 0.2 | 80 | 8.2 ± 0.05 | 2.05 |
| A5 | 0 | 8 | 0.2 | 83 | 8.9 ± 0.05 | 2.07 |

*Volume mean diameters, measured using a Sympatec ™ apparatus at 2 bar shear pressure, representing the average of two analyses.

The Table 3 data show that using the fluid inlet arrangement of the present invention, with sub-sonic anti-solvent velocities, can give yields and particle sizes comparable to, and in cases better than, those achieved using (also with sub-sonic anti-solvent velocities) the two-component coaxial nozzle of WO-95/01221. Particle size distributions can also be narrower using the present invention.

Moreover the reproducibility of the method of the invention, in terms of the product particle size, appears to be better than when using the two-component coaxial nozzle.

### EXAMPLES D

Examples A were repeated, using the same nozzle 21 but with a 0.4 mm diameter outlet.

The vertical distance y between the nozzle outlet 22 and the solution tube outlet was varied between 4 and 8 mm. The solution tube outlet was positioned in line with the nozzle outlet (ie, x=0 mm).

The supercritical carbon dioxide anti-solvent was pumped at a flow rate of 200 ml/min. The salmeterol solution flow rate was 4 ml/min. The vessel temperature and pressure were as in Examples A, and the CO₂ velocity at the nozzle outlet 22 was sub-sonic throughout the experiments. The run time for each experiment was approximately one hour.

Product particle sizes were measured using a Sympatec™ apparatus at 2 bar shear pressure.

The results are shown in Table 4.

TABLE 4

| | | Yield | Particle | Particle |
|---|---|---|---|---|
| Exp^{t}. | | (% | size | size |
| no. | y (mm) | w/w) | (μm)* | spread** |
| D1 | 4 | 49 | 11.75 ± 0.05 | 2.25 |
| D2 | 6 | 49 | 8.6 ± 0.03 | 2 |
| D3 | 8 | 49 | 8.65 ± 0.01 | 2.04 |

*Volume mean diameter, representing the average of two analyses.
**Particle size spread is defined as (D₉₀ − D₁₀)/D₅₀ where D is the volume mean diameter of the relevant particle population.

The Table 4 data indicate a preferred solution outlet position, in terms of product particle size and spread, at about y=7 mm (17.5 times the nozzle outlet diameter in this case). Again particle sizes appear to increase with both smaller and larger values of y.

1. A method for preparing a target substance in particulate form, the method comprising introducing into a particle formation vessel, through separate first and second fluid inlets respectively, (a) a solution or suspension of the target substance in a fluid vehicle (the “target solution/suspension”) and (b) a compressed fluid anti-solvent for the substance, and allowing the anti-solvent fluid to extract the vehicle from the target solution/suspension so as to form particles of the target substance, wherein the target solution/suspension enters the vessel downstream of the point of entry of the anti-solvent fluid and at a to point which lies on or close to the main axis of anti-solvent flow out of the second fluid inlet, and wherein the anti-solvent fluid has a sub-sonic velocity as it enters the particle formation vessel.

2. A method according to claim 1, wherein the target solution/suspension and the anti-solvent meet immediately downstream of the point of anti-solvent entry into the particle formation vessel.

3. A method according to claim 1 wherein the target solution/suspension and the anti-solvent meet within 0.001 to 0.1 milliseconds of the anti-solvent entering the particle formation vessel.

4. A method according to claim 1, wherein the Mach number of the anti-solvent fluid as it enters the particle formation vessel (ie, the ratio of its actual speed to the speed of sound in that fluid at that point) is 0.7 or lower.

5. A method according to claim 4, wherein the Mach number of the anti-solvent fluid as it enters the vessel is from 0.05 to 0.5.

6. A method according to claim 1, wherein the anti-solvent is a supercritical or near-critical fluid.

7. A method according to claim 1, wherein the anti-solvent fluid is carbon dioxide.

8. A method according to claim 1, wherein the target substance comprises a pharmaceutical or pharmaceutical excipient.

9. A method according to claim 1, wherein the second fluid inlet comprises a nozzle.

10. A method according to claim 9, wherein the nozzle is a convergent nozzle which focuses the anti-solvent flow through a smaller area outlet.

11. A method according to claim 1, wherein the outlet of the second fluid inlet has a diameter from 0.1 to 0.5 mm.

12. A method according to claim 1, wherein the target solution/suspension is introduced into the particle formation vessel with a back pressure.

13. A method according to claim 1, wherein the target solution/suspension is introduced directly into the anti-solvent flow, and meets with the anti-solvent flow at the point where the target solution/suspension enters the vessel.

14. A method according to claim 1, wherein the separation between (a) the outlet of the first fluid inlet and (b) the main axis of anti-solvent flow (measured in a plane perpendicular to that axis) is no more than 10 times the diameter of the outlet of the second fluid inlet.

15. A method according to claim 1, wherein the centers of the outlets of the first and second fluid inlets are both positioned on the central longitudinal axis of the second fluid inlet and/or along the main axis of anti-solvent flow.

16. A method according to claim 1, wherein the outlet of the first fluid inlet is located vertically below that of the second fluid inlet, and the anti-solvent fluid flows into the particle formation vessel in a vertically downwards direction.

17. A method according to claim 1, wherein the separation between the outlets of the first and second fluid inlets is from 15 to 25 times the diameter of the outlet of the second fluid inlet.

18. A method according to claim 1, wherein at the point where the target solution/suspension and the anti-solvent meet, the angle between their axes of flow is from 70 to 110°.

19. A method according to claim 1, wherein the first fluid inlet has an outlet of diameter less than 0.2 mm.

20. A method according to claim 19, wherein the first fluid inlet has an outlet of diameter less than 0.1 mm.

21. A method according to claim 20, wherein the first fluid inlet comprises an outlet tube section having an outlet diameter of less than 0.1 mm, mounted in fluid communication with a wider diameter tube section through which the target solution/suspension may be directed from its source towards the outlet tube section and then to the particle formation vessel.

22. A method according to claim 1, wherein the outlet of the first fluid inlet has a smaller cross sectional area than that of the second fluid inlet.

23. A method according to claim 22, wherein the outlet of the first fluid inlet has a cross sectional area which is less than 50% as large as that of the second fluid inlet.

24. A method according to claim 23, wherein the outlet of the first fluid inlet has a cross sectional area which is less than 2% as large as that of the second fluid inlet.

25. A method according to claim 1, wherein the volumetric ratio of the target solution/suspension flow rate to the anti-solvent flow rate (both measured at or immediately prior to the two fluids coming into contact with one another) is from 0.005 to 0.2.

26. A method according to claim 1, which additionally involves collecting the particles following their formation, within the particle formation vessel.
